(19) United States
(12) Patent Application Publication
(10) Pub. No.: US 2023/0373896 A1
(43) Pub. Date: Nov. 23, 2023

(54) PURIFICATION OF FERULIC ACID

(71) Applicant: RHODIA OPERATIONS, Lyon (FR)

(72) Inventors: Laurent Garel, Lyon (FR); Tiago Alves Mendes, Solaize (FR)

(73) Assignee: RHODIA OPERATIONS, Lyon (FR)

(21) Appl. No.: 18/247,606

(22) PCT Filed: Sep. 30, 2021
(86) PCT No.: PCT/EP2021/076973

§ 371 Date: Mar. 31, 2023

(30) Foreign Application Priority Data
Oct. 2, 2020 (FR) 2010088
Jul. 5, 2021 (EP) 21183653.1

(51) Int. Cl. C07C 51/42

(52) U.S. Cl. CPC C07C 51/42

(57) ABSTRACT

The present invention relates to a method for purifying a liquid medium comprising ferulic acid, a solvent and at least one impurity, the method comprising a step (a) of bringing the liquid medium into contact with a first complexing agent so as to obtain a first precipitate P1 and a liquid medium C1, and a step (b) of separating the first precipitate P1 from the liquid medium C1 so as to obtain a liquid medium F1 comprising purified ferulic acid. The present invention also relates to a method for separating ferulic acid oligomers and to their use as a scavenger of free radicals or radical species, in particular as a polymerisation inhibitor, antioxidant or UV stabiliser.

The present application claims the priority of the patent applications filed on Oct. 2, 2020 under number FR 2010088 and Jul. 5, 2021 under number EP21183653.1, the content of which is incorporated in full by way of reference.

### TECHNICAL FIELD

The present invention relates to a process for purifying a liquid medium comprising ferulic acid, a solvent and at least one impurity. The invention also relates to a composition comprising purified ferulic acid exhibiting a purity of greater than or equal to 90%. In addition, the invention relates to a process for the production of natural vanillin employing ferulic acid purified according to the process of the invention. The present invention also relates to a process for separating oligomers of ferulic acid and to the use thereof as free radical scavenger or scavenger of radical species, especially as polymerization inhibitor, antioxidant or UV stabilizer.

The invention has applications in particular in the field of food, cosmetics and flavorings.

### PRIOR ART

Ferulic acid or 3-(4-hydroxy-3-methoxyphenyl)prop-2-enoic acid is an antioxidant naturally present in plants and in particular cereals, such as rice, corn, wheat or oats. It may also be present in solid or liquid coproducts of the food-processing industry, in particular the oil-producing, cereal, sugar or alcohol sectors.

Ferulic acid may be prepared by chemical synthesis or by a biotechnological route in which a microbial fermentation or a plant tissue culture is involved. It may also be obtained by a route described as natural and/or biosourced in which a plant material is treated in order to extract ferulic acid from said plant material. For example, it may be extracted from byproducts of the food-processing industry or from grains, for example according to the process described in WO2014/187784.

Ferulic acid is used in various fields ranging from cosmetics to the food industry, in particular in the preparation of a very widely consumed flavoring substance, vanillin.

Vanillin may be produced by chemical synthesis; however, natural flavorings are preferred to synthetic flavorings by consumers. In order to meet current demand, a particular interest has been directed at the preparation of non-synthetic vanillin. Thus, growth has been seen in methods for the preparation of natural vanillin using natural and/or biosourced materials, it being possible for these methods to be described as natural according to current legislation.

In particular, natural vanilla may be obtained by a biotechnological process comprising in particular the culturing of a microorganism capable of making possible the bioconversion of a fermentation substrate into vanillin. Such a biotechnological process is, for example, described in the application EP 0 885 968, in which a microorganism converts ferulic acid into vanillin. The natural vanillin thus obtained generally undergoes steps of extraction and/or of purification. For example, the vanillin may be purified according to the methods described in the applications WO2014/114590, EP 2 791 098 or WO2018/146210.

Biosourced ferulic acid, in particular that available commercially, may exhibit an unsatisfactory degree of purity. The impurities present in the ferulic acid provided by suppliers are generally unidentified and may influence the effectiveness of the industrial processes in which the ferulic acid is employed. For this reason, in order to obtain better production yields, of natural vanillin in particular, it is desirable to have available a starting material having a high purity.

The document WO2004/110975 describes the treatment of the liquor from the cooking with lime of the maize grain resulting in an effluent known as nejayote containing ferulic acid, the treatment comprising a filtration, an acidification, the adsorption of the ferulic acid on a matrix and then the washing of the matrix and the elution with an organic solvent. The ferulic acid recovered is subsequently subjected to an additional step of recrystallization.

The document CN104628553 describes the purification of an alkaline solution of ferulic acid in which the solution is subjected to successive passes through membrane separation systems which may bring about losses of ferulic acid and a decrease in the yield of ferulic acid. The permeate subsequently obtained is acidified in order to precipitate the ferulic acid, which is recovered by centrifuging or filtration. The ferulic acid obtained is subjected to an additional step of purification over activated carbon and a purification by recrystallization.

The document EP 3 612 511 describes the extraction and the purification of ferulic acid from biomass resulting from agriculture. The ferulic acid extracted may be purified by extraction methods employing an organic solvent.

### BRIEF DESCRIPTION OF THE INVENTION

It is an aim of the present invention to provide a simple and effective solution which makes it possible to have available ferulic acid having a purity sufficient for use in fermentation, directly or indirectly.

Furthermore, the impurities present in solid or liquid ferulic acid compositions contribute to the fouling of the industrial plant with which the composition is in contact. In particular, these impurities may result in difficulties in employing ferulic acid in industrial-scale processes. For example, the impurities which are present in the solid form may result in deposits, partial or complete blockages, or fouling events which make it difficult to carry out the process and result in losses in yield, in productivity or in quality of the final product. In addition, the impurities may react with reactants, intermediates or final products of the conversion of ferulic acid into an advantageous product. The result of this is that the presence of impurities results in losses and in a fall in the production yield, for example of vanillin.

There thus exists a need to have available a simple and economically viable process for purifying ferulic acid, in particular natural or biosourced ferulic acid.

To this end, it is an aim of the invention to provide a process for the purification of ferulic acid, in particular of biosourced ferulic acid, with a high yield of ferulic acid, in particular of greater than 90%. The purified ferulic acid obtained according to the invention may be natural and biosourced, contained in a liquid medium or in the solid form.

To this end, a first aspect of the invention provides a process for purifying a liquid medium C comprising ferulic acid, a solvent and at least one impurity, the process comprising:
a step (a) of bringing the liquid medium C into contact with a first complexing agent, so as to obtain a first precipitate and a liquid medium C1, and
a step (b) of separation of the first precipitate P1 from the liquid medium C1, so as to obtain a liquid medium F1 comprising purified ferulic acid. Step (b) also makes it possible to obtain a first precipitate P1.

Embodiments, taken in isolation or in combination, additionally make provision for:
the ferulic acid to be a biosourced ferulic acid,
the liquid medium C1 to comprise purified ferulic acid exhibiting a purity of greater than 90%, preferably of greater than 95%,
the process to comprise a step (a1) in which the liquid medium C1 obtained subsequent to step (a) is heated to a temperature of between 40° C. and 70° C., preferably to 50° C.,
the pH of the liquid medium obtained in step (a) and/or (a1) to be between 6 and 9, preferably between 6 and 7,
the first complexing agent to be a divalent or trivalent cation chosen from the group consisting of transition metals, metals or alkaline earth metals or a mixture of these,
the first complexing agent to be chosen from aluminum or zinc,
the process to comprise a step (c) of addition of a second complexing agent to the liquid medium (F1), so as to obtain a second precipitate and a liquid medium (C2),
the second precipitate P2 to be distinct from the first precipitate P1,
the second complexing agent to be chosen from phosphate, dihydrogenphosphate or diphosphate ions,
the process to comprise a step (d) of separation of the second precipitate P2 from the liquid medium (C2), so as to obtain a liquid medium (F2) comprising the purified ferulic acid,
the solvent to be water and the liquid medium to be an aqueous liquid medium,
the process to comprise a step of recovery of the ferulic acid in the solid form in which the liquid medium F1 or the liquid medium F2 is brought to a pH of between 1 and 4, and the solid ferulic acid is separated from the liquid medium.

In a second aspect, the invention relates to an aqueous composition of purified biosourced ferulic acid capable of being obtained by the process as defined above; preferably, the purified ferulic acid exhibits a purity of greater than or equal to 90%, more preferentially still of greater than or equal to 95%.

According to another aspect, the invention relates to a process for the production of natural vanillin comprising—the purification of a liquid medium comprising ferulic acid according to the above process—the conversion of the purified ferulic acid obtained into natural vanillin by a fermentation process.

Advantageously, the process according to the invention comprises few steps, thus limiting the losses of ferulic acid.

The present invention is also directed to a process for purifying impurities present in solid or liquid ferulic acid compositions. Specifically, these impurities may be reused in various applications in the food and cosmetics industry, fuel stabilizers, unsaturated monomers, or polymers. These compounds may in particular be used as free radical scavengers or scavengers of radical species, especially, in particular, as polymerization inhibitors, antioxidants or UV stabilizers.

The reuse of these impurities improves the economic yield of the process of the present invention by making it possible on the one hand to purify the ferulic acid but also to reuse the impurities present in the solid or liquid ferulic acid compositions.

Thus, the present invention relates to a process for extracting oligomers of ferulic acid present in the precipitate P1.

According to another aspect, the present invention relates to the use of a precipitate P1 or of oligomers of ferulic acid present in the precipitate P1, or obtained by the extraction process of the present invention, in the field of the food or cosmetics industry, fuel stabilizers, unsaturated monomers, or polymers, especially as polymerization inhibitors, antioxidants or UV stabilizers.

### DESCRIPTION OF THE FIGURES

Other characteristics and advantages of the invention will become more apparent on reading the description which will follow. The latter is purely illustrative and should be read with regard to the appended drawings, in which:

FIG. 1 is a scheme showing a diagram of steps illustrating an embodiment of the process.

FIG. 2 illustrates an implementational example of the process according to one embodiment.

FIG. 3 illustrates the free-radical-scavenger properties of the oligomers of ferulic acid compared to a Trolox control.

### DETAILED DESCRIPTION

In the context of the present invention, and unless otherwise indicated, the expression “between x and y” includes the values x and y. In the context of the present invention, and unless otherwise indicated, the term “ppm” means “parts per million”. This unit represents a fraction by weight: 1 ppm=1 mg/kg.

A subject matter of the present invention is a process for the purification of a liquid medium C comprising ferulic acid, a solvent and at least one impurity, the process comprising at least one step (a) of bringing the liquid medium C into contact with a first complexing agent.

Ferulic acid of natural origin corresponds to the following formula:

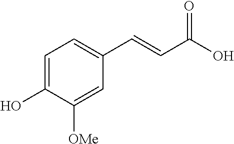 [SMILES: COc1cc(/C=C/C(=O)O)ccc1O]

Ferulic acid, when it is biosourced, may be denoted as “natural product”. According to the regulations in Europe and in the United States, this means that the product is obtained by physical, enzymatic or microbiological processes starting from plant or animal materials. Biosourced ferulic acid is understood to mean ferulic acid entirely or significantly of plant or marine origin. For example, biosourced ferulic acid may result from agricultural byproduct, plants, seeds, forest materials or algae. In particular, biosourced ferulic acid is of plant origin. Thus, biosourced ferulic acid does not result from a chemical synthesis.

The process of the present invention consists in purifying a liquid medium C comprising crude ferulic acid, the medium containing at least one unidentified impurity and a solvent. The process of the present invention consists in purifying a liquid medium C comprising crude ferulic acid, the medium containing at least one unidentified and/or known impurity and a solvent. The medium C to be purified is also denoted “starting medium or composition C” or “initial medium C”.

The initial liquid medium C may be prepared by mixing commercial crude ferulic acid in the solid form with a solvent. The process then comprises a preliminary step of preparation of the liquid medium C in which the starting ferulic acid in the solid form is brought into contact with a solvent; preferably, the solvent consists of water.

Preferably, the crude ferulic acid in the solid form to be purified or in the liquid medium C exhibits a purity of less than or equal to 87%, preferably of less than or equal to 85%, more preferentially of less than or equal to 83%. Generally, the initial crude ferulic acid exhibits a purity of greater than or equal to 60%, preferably of greater than or equal to 70%, more preferentially of greater than or equal to 80%.

Preferably, the solvent of the medium C consists of water.

According to one embodiment, the ferulic acid in the solid form to be purified is biosourced. For example, the biosourced ferulic acid is a ferulic acid obtained by treatment of all or part of maize grains or of rice or of oat grains. By way of example, the biosourced ferulic acid may be produced according to the process described in the document WO2004/187784.

According to a preferred embodiment, the initial crude ferulic acid is a natural ferulic acid.

Subsequent to the mixing of the ferulic acid in the solid form and the solvent, the starting medium C may be obtained in the form of a suspension comprising a liquid phase and a solid phase insoluble in said solvent.

The crude ferulic acid may be in the form dissolved in the liquid phase of the medium C and/or may be contained in the solid phase of the liquid medium.

The impurities may be in the form dissolved in the liquid phase of the medium C and/or may be in the solid phase of the liquid medium C.

The liquid medium C may also be a liquid composition resulting from a process for the extraction of ferulic acid in which at least one plant material has been treated, such as that described in the application WO2004/187784, that described in the application WO 2001/06789, or according to the document WO04110975A1.

The total impurities/ferulic acid ratio by weight in the initial liquid medium C is generally between 0.05 and 0.5, preferably 0.20 and 0.25. Thus, the process according to the invention is especially targeted at removing all or a portion of these impurities in order to improve the purity of the initial ferulic acid. In some cases, these impurities may be reused in various applications.

According to the process of the invention, the liquid medium C comprising ferulic acid to be purified is brought into contact with a first complexing agent. “Complexing agent” is understood to mean a substance capable of generating a precipitate insoluble in the solvent of the liquid medium, in particular insoluble in water.

According to one embodiment, the first complexing agent is a cation, in solution in a solvent, preferably in water or in a solvent mixture.

For example, the first complexing agent is advantageously in the form of a solution of monovalent, divalent, trivalent, tetravalent or pentavalent cation salt, in particular a divalent or trivalent cation salt.

The cation, in particular divalent or trivalent cation, salt may be a sulfate, chloride, nitrate, carbonate, phosphate, hydroxide or acetate salt or a mixture of these.

The cation, in particular divalent or trivalent cation, may be chosen from the group consisting of transition metals, metals, alkaline earth metals and rare earth metals, it being understood that the cation, when it is brought into contact with the starting medium C, is capable of forming a precipitate insoluble in the solvent of the medium C, in particular in water.

In one embodiment, the first complexing agent is a cation of a transition metal chosen from the group consisting of iron, nickel, copper, titanium, zirconium or a mixture of these, preferably chosen from iron or copper.

According to one embodiment, the first complexing agent is a metal cation chosen from the group consisting of aluminum and zinc.

According to another embodiment, the first complexing agent is a cation of an alkaline earth metal chosen from the group consisting of calcium and magnesium.

According to an alternative form, the first complexing agent is chosen from the group consisting of rare earth metals, such as yttrium or lanthanides, or metal oxides, such as Al₂O₃, TiO₂, SiO₂ and/or ZnO.

When the liquid medium C is obtained from crude ferulic acid in the solid form, the amount of the first complexing agent added to the liquid medium C may be greater than or equal to 1%, preferably greater than or equal to 5%, by weight, with respect to the total weight of the starting crude ferulic acid in the solid form. According to one aspect, the amount of the first complexing agent is less than or equal to 12%, preferably less than or equal to 10%, by weight, with respect to the total weight of the starting crude ferulic acid in the solid form.

Thus, the first complexing agent forms a complex with one or more impurities of the liquid medium C to form a first precipitate. Advantageously, the first complexing agent interacts selectively with one or more impurities of the medium C but does not interact or only slightly interacts with the ferulic acid present in the medium C.

Subsequent to bringing the medium C into contact with the first complexing agent, a first precipitate P1 is formed.

The medium obtained after the addition of the first complexing agent to the liquid medium C is referred to as “liquid medium C1”. The medium C1 comprises a solvent or a solvent mixture, the ferulic acid, the first precipitate P1 and at least one impurity.

Thus, step (a) of bringing the liquid medium C into contact with a first complexing agent makes it possible to obtain a first precipitate P1 and a liquid medium C1, the liquid medium C1 comprising the first precipitate P1.

The solvent of the liquid medium C1 may comprise the solvent of the starting medium C and optionally the solvent of the first complexing agent. In a preferred embodiment, the solvent comprises or consists of water.

The ferulic acid of the medium C1 may be in the form dissolved in the solvent of the medium C1 and/or may be in the solid phase of the liquid medium C1.

The known or unidentified impurities may be in the form dissolved in the solvent of the medium C1 and/or may be in the precipitate of the liquid medium C.

Without wishing to be committed to any one theory, the inventors are of the opinion that the first precipitate P1 is constituted partially or completely of a complex formed between at least one cation (first complexing agent) and at least one impurity. In particular, the complex formed is a complex comprising a divalent or trivalent cation. Furthermore, the inventors are of the opinion that the at least one impurity is a lignocellulose material chosen from the group consisting of lignin, cellulose, lignocellulose and hemicellulose.

In an implementational example, the at least one impurity is an oligomer of hydroxycinnamic acids, such as a dimer, trimer or tetramer of ferulic acid or a mixture of these. Surprisingly, the first complex is formed in a selective manner between the first complexing agent and at least one impurity, with respect to the ferulic acid present in the starting liquid medium C.

Lignocellulose material is understood to mean a material containing cellulose, hemicellulose or lignin.

Lignin is a macromolecular compound and a major structural component of the walls of plant cells. The dimer of ferulic acid may be a dihydroferulic acid of following structure:

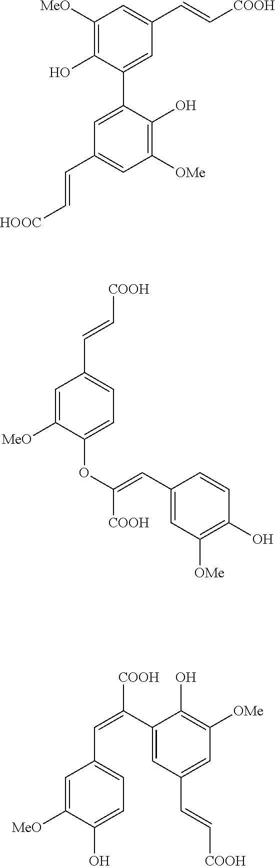 [SMILES: COc1cc(/C=C/C(=O)O)cc(-c2cc(/C=C/C(=O)O)cc(OC)c2O)c1O ; COc1cc(/C=C(/Oc2ccc(/C=C/C(=O)O)cc2OC)C(=O)O)ccc1O ; COc1cc(/C=C(/C(=O)O)c2cc(/C=C/C(=O)O)cc(OC)c2O)ccc1O]

The trimer of ferulic acid may be a dehydrotriferulic acid of following structure:

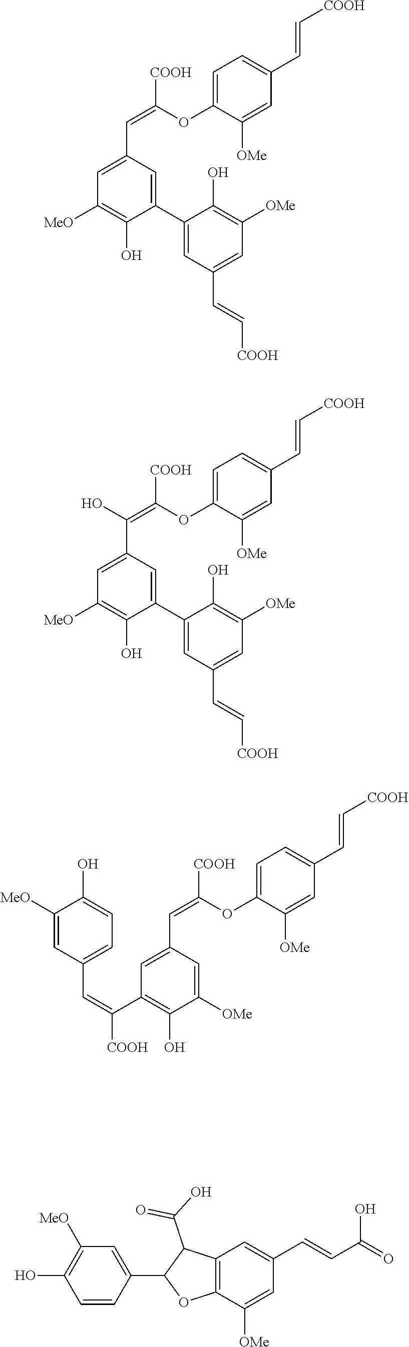 [SMILES: COc1cc(/C=C/C(=O)O)ccc1O/C(=C\c1cc(OC)c(O)c(-c2cc(/C=C/C(=O)O)cc(OC)c2O)c1)C(=O)O ; COc1cc(/C=C/C(=O)O)ccc1O/C(C(=O)O)=C(/O)c1cc(OC)c(O)c(-c2cc(/C=C/C(=O)O)cc(OC)c2O)c1 ; COc1cc(/C=C(/C(=O)O)c2cc(/C=C(\Oc3ccc(/C=C/C(=O)O)cc3OC)C(=O)O)cc(OC)c2O)ccc1O ; COc1cc(C2Oc3c(OC)cc(/C=C/C(=O)O)cc3C2C(=O)O)ccc1O]

The tetramer of ferulic acid may be a dehydrotetraferulic acid of following structure:

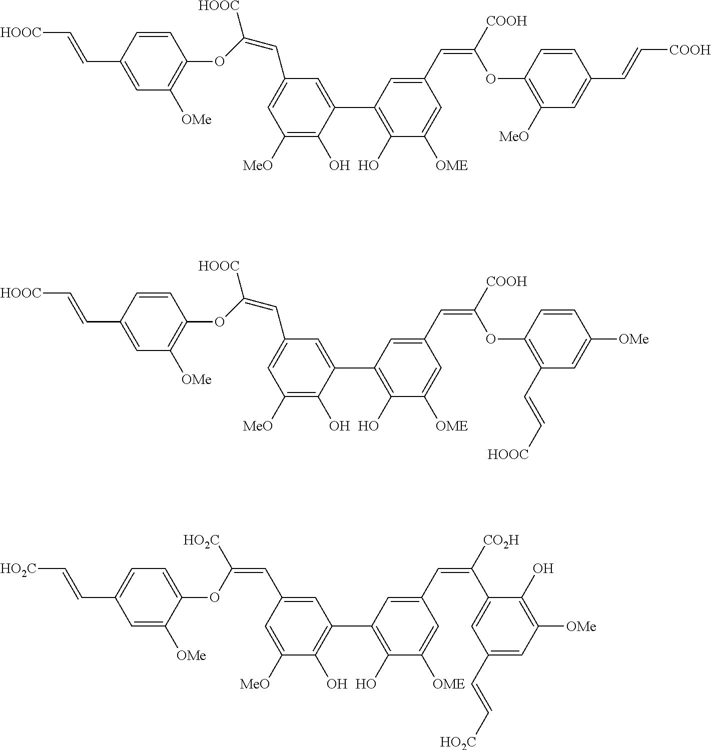

Thus, in particular, the liquid medium C comprises ferulic acid, a solvent and at least one impurity chosen from the group consisting of lignin and lignocellulose. Preferably, the ferulic acid exhibits a purity of greater than or equal to 80%.

According to an alternative form, the liquid medium C comprises ferulic acid, a solvent and at least one impurity chosen from the group consisting of dimers, trimers and tetramers of ferulic acid. Preferably, the ferulic acid exhibits a purity of greater than or equal to 80%.

Furthermore, the inventors have demonstrated that the impurities present in the liquid medium C may comprise coumaric acid.

Thus, the liquid medium C as defined above may additionally comprise coumaric acid.

According to an alternative form, the liquid medium C1 comprises ferulic acid, a solvent or a mixture of solvent and of coumaric acid.

Coumaric acid of natural origin corresponds to the following formula:

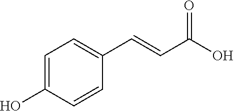 [SMILES: O=C(O)/C=C/c1ccc(O)cc1]

The inventors have found that the liquid medium C may comprise at least 0.5% of coumaric acid, for example at least 0.9% of coumaric acid, in particular less than 2% of coumaric acid, by weight, with respect to the total weight of the initial crude ferulic acid in the solid form.

According to one embodiment, the starting liquid medium C comprises at least 5% of lignin, for example more than 12% of lignin, in particular less than 16% of lignin, by weight, with respect to the total of crude ferulic acid in the solid form.

According to one aspect, the starting liquid medium C comprises at least 5% of dimers, trimers and/or tetramers of ferulic acid, for example more than 12%, in particular less than 16% of dimers, trimers and/or tetramers of ferulic acid, by weight, with respect to the total weight of initial crude ferulic acid in the solid form.

According to a preferred embodiment, in a step (a1), the liquid medium C1 obtained in step (a) is heated to a temperature of between 40° C. and 70° C., preferably to a temperature of between 50° C. and 60° C. Advantageously, when the operation is carried out in the temperature range according to the invention, it is possible to prevent the degradation of the ferulic acid. The container in which the medium C1 is contained may be heated by suitable heating means, such as a heat-exchange fluid.

According to a preferred embodiment, the pH of the liquid medium C1 is adjusted and is between 6 and 9, preferably between 6.5 and 7.5. The pH may be adjusted with a base, preferably a strong base. Advantageously, the base is chosen from water-insoluble inorganic bases. In particular, the base is chosen from the group consisting of alkali metal hydroxides, alkaline earth metal hydroxides, alkali metal hydrogencarbonates, alkaline earth metal hydrogencarbonates and their mixtures. Preferably, the base is chosen from NaOH, KOH, CaO, Ca(OH)₂ and Na₂CO₃. In addition, the adjustment of the pH makes it possible to obtain the ferulic acid in the ferulate form and thus to dissolve the ferulic acid in the liquid medium, in particular in an aqueous liquid medium.

The first precipitate P1 formed in step (a) is subsequently separated from the liquid medium C1, in a step (b), in order to result in a liquid medium F1, on the one hand, and in the first precipitate P1, on the other hand.

Preferably, the solid/liquid separation step (b) is carried out under warm conditions, the liquid medium C1 being heated to between 40° C. and 70° C. and exhibiting a pH of between 6 and 9.

The liquid medium F1 comprises the purified ferulic acid, one or more solvents and at least one impurity in a reduced amount, said impurity being chosen from the group consisting of dimers, trimers and/or tetramers of ferulic acid, coumaric acid and lignin. The liquid medium F1 is a homogeneous liquid medium, and preferentially the solvent comprises water or consists of water.

“Purified ferulic acid” is understood to mean ferulic acid which is solid or in a liquid medium, said acid being at least partially freed from at least one impurity. The purified ferulic acid is thus a ferulic acid comprising a reduced amount of impurities, by weight or by moles, with respect to the starting crude ferulic acid. Thus, the purified ferulic acid is characterized by a greater purity in comparison with the purity of the starting ferulic acid.

Advantageously, after step (b), the dry weight of at least one impurity is decreased at least by half, with respect to the dry weight of the same impurity in the medium C.

Advantageously, the purity of the ferulic acid, in particular biosourced, in the liquid medium F1 is greater than or equal to 87%, preferably greater than or equal to 90%, more preferentially greater than or equal to 95%.

The liquid medium F1 also contains, in a residual manner, the first complexing agent in the cation form.

The residual content by weight of the first complexing agent (cation) in the liquid medium F1 is greater than or equal to 5 ppm, preferably greater than or equal to 300 ppm, more preferentially greater than or equal to 400 ppm, with respect to the weight of the medium F1. According to one aspect, the residual content by weight of the first complexing agent in the liquid medium F1 is less than 2000 ppm, with respect to the weight of the medium F1.

Thus, on conclusion of step (b) of the process according to the invention, a purified, preferably biosourced, ferulic acid is obtained from an impure solution of ferulic acid.

According to an alternative form, the process relates to a process for the purification of a liquid medium comprising ferulic acid having a degree of purity of greater than or equal to 80%, the process comprising:
a step (a) of obtaining a first precipitate P1 and a liquid medium C1, the liquid medium C1 comprising a solvent, ferulic acid, the first precipitate P1 and at least one impurity, for example coumaric acid,
a step (a1) of heating the liquid medium C1 obtained in step (a), the medium C1 being brought to a temperature of between 40° C. and 70° C., the pH of the liquid medium C1 being between 6 and 9, and
a step (b) of separation of the first precipitate P1 and of the warm medium C1, so as to obtain a liquid medium F1 comprising the purified ferulic acid.

In a specific embodiment of the invention, the process additionally comprises a step (c) in which a second complexing agent is added to the liquid medium F1. Thus, step (c) is subsequent to step (b) of isolation of the precipitate.

The second complexing agent may be an anion chosen from the group consisting of phosphate PO₄³−, dihydrogenphosphate H₂(PO₄)⁻, diphosphate P₂O₇⁴⁻, oxalate (COO)₂²⁻ and vanadate VO₄³⁻ ions and their mixture. Advantageously, the second complexing agent is in solution, preferably in aqueous solution.

Generally, the second complexing agent/residual first complexing agent molar ratio is between 1 and 3, preferably between 1.1 and 1.8.

Subsequent to the addition of the second complexing agent to the medium F1, a second precipitate P2 is formed. The second precipitate P2 is distinct from the first precipitate P1. In addition, a liquid medium C2 comprising the second precipitate P2, a solvent, purified ferulic acid and at least one impurity chosen from dimers, trimers and/or tetramers of ferulic acid, coumaric acid and lignin is obtained. The solvent of the liquid medium C2 may comprise the solvent of the starting medium C and optionally the solvent of the first complexing agent and/or the solvent of the second complexing agent. In a preferred embodiment, the solvent of the medium C2 comprises or consists of water.

Subsequent to step (c) of formation of the second precipitate P2, the process may comprise a step (d) of separation of the second precipitate P2 from the liquid medium C2 in order to obtain, on the one hand, the second precipitate P2 and, on the other hand, a liquid medium F2.

The liquid medium F2 comprises a solvent or a solvent mixture, purified ferulic acid and at least one residual impurity, for example dimers, trimers and/or tetramers of ferulic acid, coumaric acid and lignin. The solvent of the liquid medium F2 may comprise the solvent of the starting medium C, the solvent of the first complexing agent and/or the solvent of the second complexing agent. In a preferred embodiment, the solvent of the medium F2 comprises or consists of water. Advantageously, the inventors have found that the addition of the second complexing agent makes it possible, after separation of the second precipitate, to obtain a liquid medium F2 which may be used directly in a process for the preparation of vanillin by ferulic acid fermentation.

The residual content of the first complexing agent in the liquid medium F2 is greater than or equal to 1 ppm, preferably greater than or equal to 20 ppm, more preferentially greater than or equal to 40 ppm, with respect to the weight of the medium F2. Advantageously, the residual content of first complexing agent in the composition F2 is less than 70 ppm, with respect to the weight of the medium F2.

The separation of the first precipitate P1 from the medium C1 or of the second precipitate P2 from the liquid medium C2 may be carried out by any appropriate solid/liquid separation technique, including but not limited to filtration, centrifuging, separation by settling, and the like.

According to the invention, the separation of the first precipitate P1 or of the second precipitate P2 from the liquid medium in which it is contained is carried out by filtration, for example by membrane filtration, such as ultrafiltration or microfiltration, or by filtration through sintered glass, or by industrial techniques, such as filtration with filtration cloths, filter presses or filtration cartridges.

Thus, the medium C1 is filtered and the filtrate obtained, corresponding to the medium F1, comprises ferulic acid having a purity of greater than or equal to 90%, in particular of greater than or equal to 93%.

The filtration of the medium C2 results in a filtrate corresponding to the medium F2 comprising ferulic acid having a purity of greater than or equal to 90%, in particular of greater than or equal to 93%.

According to a preferred embodiment, the liquid medium C and/or C1 and/or C2 and/or F1 and/or F2 of the process according to the invention is an aqueous liquid medium consisting of water. Advantageously, the process according to the invention makes it possible to operate in an aqueous medium and does not comprise the use of an organic solvent.

Generally, in the liquid medium, the ferulic acid is in the dissolved salified form, that is to say in the ferulate form. When the ferulic acid is in the ferulate form in the liquid medium, the process according to the invention additionally comprises a step of adjustment of the pH of the liquid medium in order to precipitate the ferulic acid. The adjustment of the pH may be carried out by addition of an acid, such as a strong acid or a Lewis acid. Preferably, the acid is chosen from the group consisting of HCl, H₂SO₄, H₃PO₄ or para-toluenesulfonic acid. The precipitated ferulic acid is separated from the liquid medium, according to the abovementioned methods, and is dried in order to obtain the purified ferulic acid in the form of a solid. The ferulic acid in the solid form obtained is dried according to conventional techniques, for example by using contact dryers at atmospheric pressure or under reduced pressure.

According to one embodiment, the invention relates to a process for purifying a liquid medium C comprising ferulic acid, a solvent and at least one impurity, the process comprising:
a step (a) of bringing the liquid medium C into contact with a first complexing agent, so as to obtain a first precipitate and a liquid medium C1, and
a step (b) of separation of the first precipitate P1 from the liquid medium C1, so as to obtain a liquid medium F1 comprising purified ferulic acid,
a step of recovery of the ferulic acid in the solid form.

Advantageously, the process according to the invention does not require a preliminary step of filtration of the starting liquid medium C, thus making it possible to minimize the losses due to an excessively large number of steps. In addition, the process according to the invention may be carried out without the use of an organic solvent and thus in a more environmentally friendly way. It is thus possible to carry out the purification process under mild conditions in order to result in a natural composition of ferulic acid.

Furthermore, advantageously, the yield of purified ferulic acid, in the medium F1 or in the medium F2 or in the solid form, is greater than or equal to 85%, preferably greater than or equal to 90%, more preferentially greater than or equal to 95%.

On conclusion of step (b), a medium F1 is isolated on the one hand and a precipitate P1 is isolated on the other.

Thus, the present invention relates to a process for extracting oligomers of ferulic acid present in the precipitate P1. The precipitate P1 in general comprises at least one oligomer of ferulic acid, generally in salified form.

Thus, the process for extracting oligomers of ferulic acid present in the precipitate P1 comprises a step (A) in which an acid is mixed with the precipitate P1. The acid makes it possible to protonate the oligomers of ferulic acid present in the precipitate P1. Preferably, the acid used is phosphoric acid H₃PO₄. The acid is used in general with a concentration by weight of between 5% and 25%, preferably of between 7% and 15%, for example of 8%, 9%, 10%, 11%, 12%, 13% or 14%. Step (A) is conducted in general at a temperature of between 15° C. and 25° C. Step (A) is conducted in general with stirring.

On conclusion of step (A), a step (B) of liquid-liquid extraction is carried out. Step (B) is conducted in the presence of a solvent, preferably a solvent in which the oligomers of ferulic acid are soluble.

Preferably, the solvent is an aprotic solvent. The solvent is generally of moderate polarity. The solvent may be ethyl acetate.

The organic phase comprising the oligomer(s) of ferulic acid is separated and recovered.

Optionally, acid may be added in order to repeat the liquid-liquid extraction. The acid may be phosphoric acid.

The organic phase thus recovered is distilled so as to evaporate the solvent.

On conclusion of step (B), a composition (O) is obtained comprising at least one oligomer of ferulic acid.

Optionally, the process for extracting oligomers of ferulic acid may comprise at least one step (i) of washing the precipitate P1, prior to step (A) described above. Step (i) is carried out in the presence of a base, preferably a strong base; in particular, the base may be NaOH or KOH.

Preferably, the base added is in solution, and the concentration of the base is in general between 10% and 50% by weight, preferably between 20% and 40% by weight; the concentration may be 30% by weight.

The pH of step (i) is in general greater than or equal to 7, preferably greater than or equal to 8, very preferentially greater than or equal to 8.2. The pH of step (i) is in general less than or equal to 10, preferably less than or equal to 9, very preferentially less than or equal to 8.5. Step (i) is preferably carried out under stirring.

Without wishing to be bound by theory, step (i) would make it possible to separate oligomers of ferulic acid from the ferulic acid present in the precipitate P1 in general in the form of a ferulate, for example zinc or aluminum ferulate.

On conclusion of step (i), a liquid medium is separated from a solid phase by filtration in a step (ii). This solid phase may be subjected to a new step (i).

Steps (i) and (ii) may be repeated 1, 2, 3, 4, 5, 6, 7, 8, 9 or 10 times, making it possible to obtain a composition O comprising at least one oligomer of ferulic acid.

Steps (A), (B), and optionally (i) and (ii) are generally conducted on conclusion of step (b) described above. Steps (A), (B), and optionally (i) and (ii) may be conducted independently of steps (c) and (d). Thus, the process may make it possible to prepare a purified ferulic acid and a composition (O) comprising at least one oligomer of ferulic acid.

The present invention also relates to a composition (O) comprising at least one oligomer of ferulic acid. Advantageously, the composition (O) is of biosourced origin.

The present invention also relates to a purified composition of ferulic acid capable of being obtained by the process according to the invention. Generally, the ferulic acid of the composition is in the salified form, that is to say in the ferulate ion form.

According to one aspect, the purified composition of ferulic acid according to the invention may comprise purified ferulic acid, a liquid phase and at least one impurity chosen from dimers, trimers and/or tetramers of ferulic acid, coumaric acid and/or lignin. For example, the purified composition of ferulic acid may be the liquid medium F1 or the liquid medium F2.

According to one aspect, the ferulic acid composition according to the invention is an aqueous liquid composition comprising between 1% and 15% of purified ferulic acid, preferably between 5% and 10% by weight, with respect to the weight of the composition.

According to one aspect, the residual content of lignin or dimers, or trimers and/or tetramers of ferulic acid in the composition is between 1500 and 9000 ppm, preferably between 2500 and 8000 ppm, with respect to the weight of the composition.

According to one aspect, the residual content of first complexing agent is between 10 ppm and 650 ppm, preferably between 70 ppm and 200 ppm, with respect to the weight of the composition.

In particular, the purified ferulic acid composition according to the invention may be in the solid form; for example, it may correspond to the solid purified ferulic acid obtained after the step of acidification of the medium F1 or of the medium F2.

According to one aspect, the aqueous purified ferulic acid composition comprises ferulic acid having a purity of greater than or equal to 90%, preferably of greater than or equal to 95%.

Advantageously, the composition according to the invention does not exhibit or exhibits little in the way of water-insoluble particles. Advantageously, the purified ferulic acid composition or the purified ferulic acid may be used, directly or indirectly, in a bioconversion fermenter in order to be converted by a microorganism into natural vanillin. Very advantageously, the composition consisting of the liquid medium F2 may be used directly in a fermenter in order to be converted into vanillin by a microorganism, for example according to the method described in EP 0 885 968. According to one aspect, the solid ferulic acid composition according to the invention exhibits a purity of greater than or equal to 85%, preferably of greater than or equal to 90%. For example, the purity may be 91%, 92%, 93%, 94%, 95%, 96%, 97% or 98%. By virtue of the process according to the invention, the items of equipment used for the production and/or the purification of vanillin exhibit a significantly reduced level of clogging. Thus, it is possible to have available a purified ferulic acid composition which may be easily employed in industry and which makes it possible to achieve a better yield during the production of vanillin in particular.

In a particularly preferred implementation of the process, the ferulic acid composition obtained is a natural ferulic acid.

The present invention also relates to a process for the preparation of ferulic acid in which the ferulic acid is purified according to the purification process as defined above.

The present invention also relates to the use of a composition of ferulic acid or the use of ferulic acid, which acid is purified and obtained by the process described above, for the preparation of vanillin, in particular of natural vanillin.

The invention also relates to a process for the production of natural vanillin comprising—the purification of a medium comprising ferulic acid according to the process as described above—the conversion of the purified ferulic acid obtained into natural vanillin by a fermentation process.

The present invention also relates to a composition of oligomers of ferulic acid that is capable of being obtained by the extraction process of the present invention; the composition may be the composition O.

The present invention also relates to the use of a precipitate P1 or of a composition of oligomers of ferulic acid present in the precipitate P1, or obtained by the extraction process of the present invention, in the field of the food or cosmetics industry, especially as antioxidant or UV stabilizer. In particular, the use of a composition O in the field of the food or cosmetics industry especially as free radical scavenger or scavenger of radical species, especially as polymerization inhibitor, antioxidant or UV stabilizer.

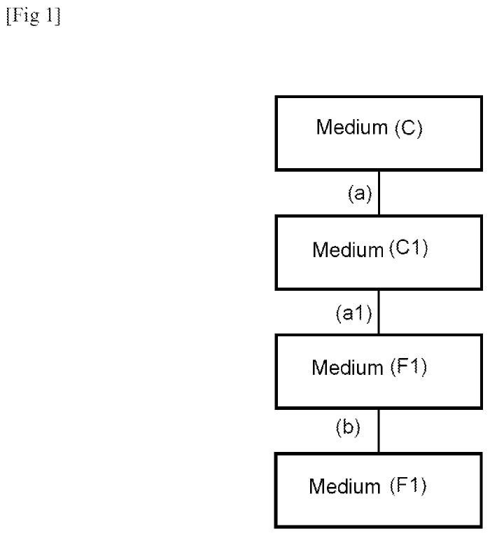

With reference to the scheme of FIG. 1, the starting liquid medium C is prepared by mixing commercial biosourced crude ferulic acid with seven times its weight of water. The starting crude ferulic acid exhibits a purity of 82% and contains identified and unidentified impurities. The mixture obtained is provided in the form of a suspension comprising the aqueous liquid medium C and solid particles suspended in the liquid medium C (not represented). In a step (a), the starting liquid medium C is brought into contact with an aqueous solution comprising the first complexing agent. Subsequent to the mixing of the initial medium C with the aqueous solution of the first complexing agent, an aqueous liquid medium C1 is obtained and a first precipitate P1 is formed. The resulting medium C1 is a suspension comprising in particular an aqueous liquid phase and the first precipitate P1. In a step (a1), the aqueous medium C1 is heated to 50° C. and then the pH of the medium C1 is brought to 6.8. In a step (b), the warm liquid medium C1 is filtered with a filtration device in order to separate and/or isolate the first precipitate P1. Optionally, the first precipitate P1 is washed with warmed water at 50° C. at alkaline pH. The filtration results in a liquid medium F1 comprising water, purified ferulic acid and at least one impurity in a reduced amount. Moreover, the first precipitate P1 is retained by the filtration device. Advantageously, the purity of the ferulate contained in the liquid medium C1 thus obtained is greater than the purity of the ferulic acid of the starting medium C. Finally, in an optional step, in order to obtain the ferulic acid in the solid form, the medium C1 is acidified to pH 2-3. The liquid of the medium C1 is separated from the solid ferulic acid obtained by filtration (not represented).

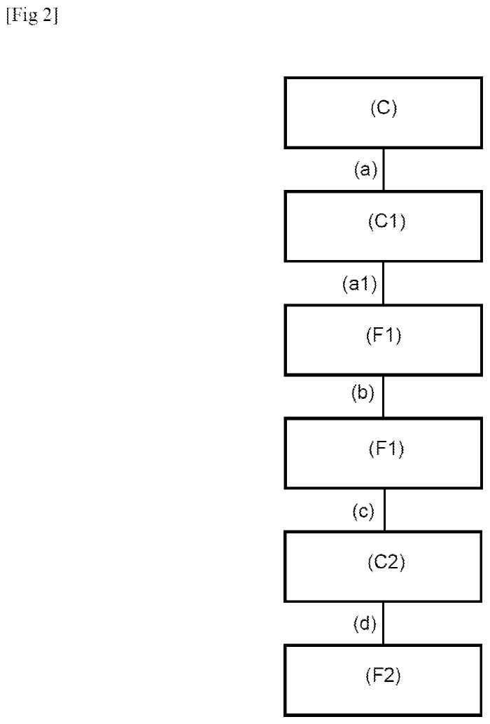

With reference to FIG. 2, steps (a), (a1) and (b) of the embodiment according to FIG. 1 are carried out. Subsequent to the filtration step (b), an aqueous liquid medium F1 is obtained. Then, in a step (c), the liquid medium F1 is brought into contact with an aqueous solution of the second complexing agent. A second precipitate P2 is then formed. The resulting composition is the medium C2 comprising the second precipitate P2, an aqueous solvent and at least one residual impurity. The medium C2 is filtered in a step (d) in order to result in the second precipitate P2, on the one hand, and in a filtrate F2, on the other hand. The filtrate is recovered and corresponds to the medium F2, F2 being a liquid medium devoid of precipitate or of particles. Advantageously, the composition F2 may be used directly in a microbiological process for the preparation of vanillin.

Should the disclosure of patents, patent applications and publications cited herein by reference conflict with the description of the present application to the extent that it risks rendering a term uncertain, the present description shall prevail.

### EXAMPLES

The examples below are intended to illustrate the invention without, however, limiting it.

### Example 1

### Purification of Commercial Biosourced Ferulic Acid

A medium C comprising ferulic acid was prepared by mixing commercial ferulic acid in the solid form (55 g) and water (409 g). The medium C obtained is stirred at ambient temperature for 30 min. 29 ml of an aqueous zinc sulfate solution (100 g/l) are added to the medium C and then the medium is brought to a temperature of 50° C. and the pH is adjusted to 6.8. The medium C1 obtained is filtered through a filtration cloth and the filtrate F1 is recovered. A liquid medium F1 comprising the purified ferulic acid is thus obtained. A precipitate P1 is isolated.

The results of example 1 are presented in table 1 below:

TABLE 1

| | Liquid medium | Medium F1 |
|---|---|---|
| | C (100 g) | (100 g) |
| Impurities/ferulic acid ratio | 0.22 | 0.09 |
| Amount of coumaric acid (g) | 0.7 | 0.6 |
| Amount of ferulic acid (g) | 44.9 | 40 |
| Purity of the ferulic acid | 82% | 92 |

### Example 2

### Comparative

Example 2 does not comprise a step of addition of a first complexing agent or of a second complexing agent. The medium C comprising ferulic acid was prepared by mixing commercial ferulic acid in the solid form (50 g) and water (413 g). The medium C obtained is stirred at ambient temperature for 30 min. The medium is brought to a temperature of 50° C. and the pH is adjusted to 6.8. The medium C1 obtained is filtered through a filtration cloth and the liquid medium F1 is recovered. A liquid medium F1 comprising the purified ferulic acid is thus obtained.

The results of example 2 are presented in the table below:

TABLE 2

| | Liquid medium | Medium F1 |
|---|---|---|
| | C (100 g) | (100 g) |
| Impurities/ferulic acid ratio | 0.22 | 0.19 |
| Amount of coumaric acid (g) | 0.6 | 0.6 |
| Amount of ferulic acid (g) | 41.4 | 40.4 |
| Purity of the ferulic acid | 82% | 84% |

Thus, as shown in table 2, the process according to the invention makes possible the purification of a liquid medium comprising ferulic acid.

### Example 3

### The Precipitate P1 of Example 1 is Recovered

The precipitate P1 is dispersed in water and the pH is adjusted to 8.2 with 30% sodium hydroxide, then filtered and dried. This new precipitate is then brought into contact with 10% by weight phosphoric acid. Extraction with ethyl acetate is then carried out.

The organic phase is dried and the solvent is evaporated. A composition (O) comprising at least one oligomer of ferulic acid is obtained.

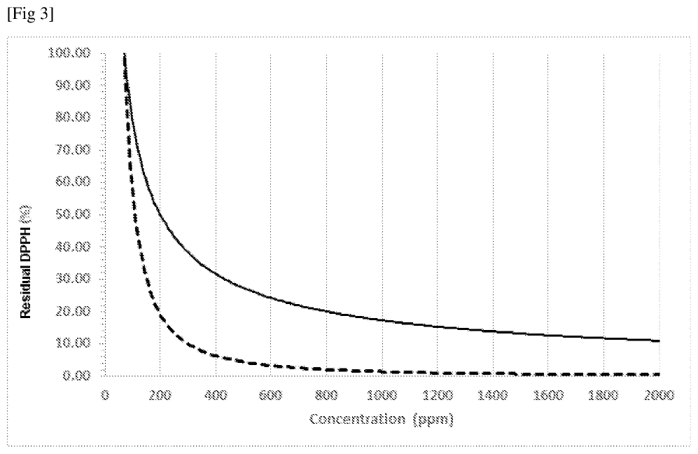

Measurements of the capacity for scavenging a radical species were carried out and are presented in table 3 and FIG. 3. Trolox is used as a control and is represented by a dotted line. The composition (O) is represented in FIG. 3 with a solid line.

TABLE 3

| Compound | Trolox | Composition (O) |
|---|---|---|
| EC₅₀ (ppm) | 109 | 200 |

The results show that the precipitate P1 and the ferulic acid oligomer composition (O) exhibit advantageous radical-species-scavenging properties. In particular, the precipitate P1 and the composition (O) have advantageous antioxidant properties.

### Protocol for Measuring the Antioxidant Power:

3 mL of a 6×10⁻⁵ mol/L DPPH* solution and 77 μL of solution of the compound of which it is desired to measure the antioxidant activity (Trolox, precipitate P1 and composition O at various concentrations) are placed in a spectrophotometer cuvette.

The absorbance at 515 nm is measured regularly over a period of 5 hours. A dose-effect curve can then be plotted and the effective concentration EC₅₀ is then determined, this measurement indicating the concentration of antioxidant required to reduce 50% of the initial DPPH°. The lower the EC₅₀, the more effective the antioxidant is.

1. A process for purifying a liquid medium C comprising ferulic acid, a solvent and at least one impurity, the process comprising:
a step (a) of bringing the liquid medium C into contact with a first complexing agent, so as to obtain a first precipitate and a liquid medium C1, and
a step (b) of separation of the first precipitate from the liquid medium C1, so as to obtain a liquid medium F1 comprising purified ferulic acid and a first precipitate P1,
in which the first complexing agent is a divalent or trivalent cation chosen from the group consisting of transition metals, metals or alkaline earth metals or a mixture of these.

2. The process as claimed in claim 1, in which the ferulic acid is a biosourced ferulic acid.

3. The process as claimed in claim 1, in which the liquid medium C1 comprises purified ferulic acid exhibiting a purity of greater than 90%.

4. The process as claimed in claim 1, comprising a step (a1) in which the liquid medium C1 obtained subsequent to step (a) is heated to a temperature of between 40° C. and 70° C.

5. The process as claimed in claim 4, in which the pH of the composition obtained in step (a) and/or (a1) is between 6 and 9.

6. The process as claimed in claim 1, in which the first complexing agent is chosen from aluminum or zinc.

7. The process as claimed in claim 1, additionally comprising a step (c) of addition of a second complexing agent to the medium F1, so as to obtain a second precipitate and a liquid medium (C2).

8. The process as claimed in claim 7, in which the second complexing agent is chosen from phosphate, dihydrogenphosphate or diphosphate ions.

9. The process as claimed in claim 7, comprising a step (d) of separation of the second precipitate from the liquid medium (C2), in order to obtain a liquid medium (F2) comprising the purified ferulic acid.

10. A process for extracting oligomers of ferulic acid present in the precipitate P1, obtained according to the process of claim 1, comprising a step (A) in which an acid is mixed with the precipitate P1.

11. The process for extracting oligomers of ferulic acid as claimed in claim 10, comprising a step (B) of liquid-liquid extraction for obtaining a composition (O) comprising at least one oligomer of ferulic acid.

12. A composition (O) comprising at least one oligomer of ferulic acid that is obtained according to claim 10.

13. (canceled)
